(19) United States
(12) Patent Application Publication
(10) Pub. No.: US 2014/0026917 A1
(43) Pub. Date: Jan. 30, 2014

(54) COSMETIC KIT, COMPOSITION FOR CHANGING THE FORM OF HAIR FIBRES AND CORRESPONDING APPLICATION METHOD

(71) Applicants: Valeria Fernandes Monteiro, Araraquara (BR); Luiz Estevao Salvagnini, Sao Carlos (BR); Monica Cristina Salvagnini, Sao Carlos (BR)

(72) Inventors: Valeria Fernandes Monteiro, Araraquara (BR); Luiz Estevao Salvagnini, Sao Carlos (BR); Monica Cristina Salvagnini, Sao Carlos (BR)

(21) Appl. No.: 14/110,297

(22) PCT Filed: Apr. 5, 2012
(86) PCT No.: PCT/BR12/00102

§ 371 Date: Oct. 7, 2013

(30) Foreign Application Priority Data
Apr. 8, 2011 (BR) PI1101698-1

(51) Int. Cl. A61K 8/66; A61K 8/92; A61K 8/46; A61K 8/97; A61K 8/37; A61K 8/41; A61Q 5/04; A45D 7/04; A61K 8/58

(52) U.S. Cl. CPC A61K 8/66; A45D 7/04; A61K 8/92; A61K 8/46; A61K 8/585; A61K 8/37; A61K 8/416; A61Q 5/04; A61K 8/975
USPC 132/205; 424/70.51

(57) ABSTRACT

The present invention relates of a Cosmetic Kit and it composition to do a conformational change of the hairs (curling or straightening) and the respective methods of application, where the composition of the kit is as follows: Cleansing Shampoo, Transformation Cream (Base cream), Enzymatic Gel, Neutralizer, Conditioner and Finishing Fluid, that can be used also in extreme fragile curly hair acting specifically in the conformational change of keratin, mainly on its disulfide bonds where the Transformation Cream (Base cream) should be used together with Enzymatic Gel that have enzymatic activators and the proteolytics enzymes E.C.: 3.4.21.14—CAS# 9014-01-1 and INS1101i (Protemax® 580 L); E.C.: 3.4.21.62—CAS# 9014-01-1 and INS1101i (Prolav® 750); and, E.C: 3.4.24.28—CAS# 76774-43-1 and INS1101i (Protemax® NL600C) that when combined act in a specific and smooth way in the fibers. The invention also presents an application method of the products that compound the Kit (a Protocol).

### FIELD OF THE INVENTION

The present invention relates of a Cosmetic Kit and it composition to do a conformational change of the hairs (curling or straightening) and the respective methods of application, where the composition of the kit is as follows: Cleansing Shampoo, Transformation Cream (Base cream), Enzymatic Gel, Neutralizer, Conditioner and Finishing Fluid, that can be used also in extreme fragile curly hair acting specifically in the conformational change of keratin, mainly on its disulfide bonds where the Transformation Cream (Base cream) should be used together with Enzymatic Gel that have enzymatic activators and the proteolytics enzymes E.C.: 3.4.21.14—CAS 9014-01-1 and INS1101i (Protemax® 580 L); E.C.: 3.4.21.62—CAS 9014-01-1 and INS1101i (Prolav® 750); and, E.C: 3.4.24.28—CAS 76774-43-1 and INS1101i (Protemax® NL600C) that when combined act in a specific and smooth way in the fibers. The invention also presents an application method of the products that compound the Kit (a Protocol).

### BACKGROUND OF THE INVENTION

The products nowadays at the market to straightening or curling of the hairs act like a reduction agents of chemical bonds that keep the structure the hair keratin. The main reduction agents are sodium hydroxide and potassium hydroxide and organic hydroxides like guanidine. The thioglycolates, triethanolamine and diethanolamine are also largely used. These products are very effective but also, extremely aggressive to the hair, scalp and skin of a customer. The formol and formaldehyde (forbidden by a Brasilian Health Bureau—ANVISA) also were very used in straightening of hair and were very toxic.

Products on the market for conformational change of capillary fibers have the chemical groups: metal hydroxides (sodium hydroxide, potassium hydroxide, lithium hydroxide), organic hydroxides (sodium hydroxide guanidine) as well as organic derivatives of ammonia (ethanolamine, triethanolamine) as well as thioglycolates (sodium, ammonium). These assets present in high concentrations which makes the final product very alkaline (pH 14), causing severe injuries to the scalp of the user as well as poisoning enforcer and user.

A patent widely used in commercial products is number PI9405526 BR-2 which relates to a hair straightening system consists of two components, which produces a highly alkaline hydroxide consisting of a cream containing a base (guanidine, N-methyl guanidine acetamide dimetilaminoamidina, and aminoamidina acetamidine) with an anion capable of being precipitated by an alkaline earth ion (carbonate, sulfate, sulfite, phosphate, fluoride, oxalate, tartrate, laurate), specifically with guanidine carbonate to a concentration of 1 to 20% in the creams.

The patent document PI0705933 BR-7 proposes a method for permanently reshaping the hair without affecting the artificial color, especially short-haired and thin by increased volume. It is proposed the use of a kit in which the hairs are initially tensioned, tensioning mechanically under the action of a tulip-containing cationic polymer product with application of a product containing reducing agent concentration of 0.1 to 3% by weight, kept from 2 until 30 minutes with pH variable from 7.5 to 11 with subsequent use of the oxidizing agent hydrogen peroxide or persulfates, variant pH between 1.5 to 4.5. This patent document also proposes the use of a kit comprising products containing cationic polymers that assist in fixing and tensioning of the hair to promote remodeling and permanent increase in volume, the required smooth and fine hair, also being specific for Caucasians hair.

Another patent document BR-4 PI0900800 A2 proposes the use of a Kit for permanently reshaping the hair, using a pre-treatment product comprising a non-reducing acid in preferred pH between 2.5 to 4.0, which is subsequently washed, and applying a treatment product containing an organic or metal hydroxide, preferably sodium hydroxide or guanidine hydroxide. The reshaping of hair may be performed before, during or after application of the treatment product. The post-treatment is optional and may be performed with product containing an inorganic or organic acid or an acid salt. Are suggested mono or polyacid derived from hydrochloric, sulfuric, phosphoric or boric acid. It is not clear in the cited patent which is the composition, action and specific use of each product, as they would make a kit.

The document BR P10900800-4 A2, involves the use of a kit for reshaping of Caucasian hair specifically looking for increased volume and hair protection by the use of a product in acid pH and the use of io reducing agents such as hydroxides. The patent is very specific to promote crimping or curly of the hairs resulting on an increased volume.

Another patent document U.S. 2010/0012142 A1, proposes the use of a proteolitic enzyme preferably the enzyme Ker A® for smoothing the hair. The patent mentions the use of a pretreatment formulation (sweeling solution), citing various components commonly used in formulations for creams, shampoos and other hair care products. Subsequently the smoothing product is applied at the hair and should be maintained in the hair for 45 minutes at 45° C. The hair is then dried and stretched by the use of thermal board. Test was performed for straightening of Caucasians hair. However, the document is not clear regarding the pretreatment formulation (sweeling solution), its function, citing several components commonly used in hair care formulations; also suggests that the study was conducted only in Caucasian hair, not indicating in all document a possibility of use in African hair.

It was considered that the pre-treatment solution should be kept on the hair for 25 minutes without informing how should be the procedure of application of the product. Subsequently it is suggest the use of straightening product containing the enzyme that should be kept on the hair for 45 minutes at 45° C. There is no guidance regarding a protocol or application method, especially how to keep the customer hair at 45° C. for 45 minutes containing the cited product. There isn't also in this patent, after the withdrawal of the solution and shaping the hair, an orientation in neutralizing the action of reducing agents (ethanolamine, triethanolamine, ammonia) used in the pretreatment solution. Subsequently it is suggested that the hair is patterned by using thermal board without any guidance for procedure of completing the treatment.

The second point refers to the time of re-application of the product shown in that patent that is 45 minutes at 45° C., besides of 25 minutes suggested for an action of the pre-treatment product. The time is too long, over 1 hour for the action of the product and, in addition there is no directions as how the hair will be kept by this time at the suggested temperature. There isn't also a protocol or method of application stating the details of the use of the product by the consumer or professional

### ADVANTAGES OF THE INVENTION TO SOLVE THE PROBLEMS DESCRIBED ABOVE

One of the differences from the Kit (object of the invention) that is now claimed is the use of proteolytic enzymes EC: 3.4.21.14—CAS# 9014-01-1 and INS1101 i, EC: 3.4.21.62—CAS# 9014-01-1 and INS1101 i, and EC: 3.4.24.28—CAS# 76774-43-1 and INS1101i, whose products are supplied by the company Prozyn Industry and Trade Ltd., presenting respectively the following trade names: Protemax® 580 L; Prolav® 750 and Protemax® NL600C.

The kit is composed by six proposed products, which have minimum amounts of active working in a minimum time and with minimum degradation of the hair fibers with desired effect.

The pre-treatment product, a cleansing shampoo, has appropriate formulation for cleaning and prepare of the hair and scalp for the treatment with the conformational modifier product. This shampoo contains in addition of surfactants, commonly used in cosmetic products, a surfactant of vegetable origin, extract of Pelvetia canaliculata, which is safe and effective in cleansing the hair. Products containing enzyme activating agents, in addition to proteolytic enzymes, act synergistically with components present in the formulations to act effectively and safely.

The concentrations of enzyme products, the pH maintenance gel, and preservatives in addition to other components of the formulations were selected according to previous tests of stability, safety and efficacy of the product. Along the gel are added enzymatic activators cysteine and cysteine hydrochloride. Mixed with the gel was proposed to formulate a base cream containing agents that enhance the action of enzymes, the enzyme activators calcium hydroxide, sodium sulfite besides of the ammonium thioglycolate which also functions as the reducing agent. The pH of the conformational mixture reaches a maximum value of 10. The Cream containing different enzymatic activators are brought separately to the Enzymatic Gel product to ensure the preservation of long shelf life products. After the action of the conformational change agent, two products were formulated to restore the integrity of the hair fibers: the Conditioner and the Finishing Fluid. The Conditioner has a number of high quality ingredients which function of to permeate through the cuticle and reaching the capillary cortex in order to maintain the new conformation acquired by the reconnection and replacement of amino acids of the hair keratin, besides the interaction of the hair, assisting the restoration of tensile strength of the fibers. After dry, the Finishing Fluid is applied with the aid of a thermal board, comprising a series of silicones with different functions: thermal protection, sealant cuticles providers, shine and smoothness to keep the fibers in the new conformation, protected from physical and chemical agents. Adding all periods required for the action of the kit, this time is a maximum of 50 minutes, all with short steps of implementation to guide the professional applies the product and without discouraging the client that uses it. With the safety test product for each type of client's hair (application into small strands, prior to using the kit) reaches maximum time of 1 hour and 10 minutes.

Thus, each of the formulations present in the kit is specific for the use of minimal amounts of reducing agents, the use of proteolytic enzymes with enzyme activators to facilitate the process of cleavage of chemical bonds of specific keratin, thus making the product safe and gentle for both client as to the applicator. The selection of assets, as well as the protocol used, ensure product effectiveness rated by institution registered with ANVISA (National Health Surveillance Agency).

### AIMS OF THE INVENTION

The product to be patented is presented as a kit and complete treatment composition of the hair fibers and conformational change in much less aggressively way than commercial products on the market, and effectively straightening or curly of the hair, taking into account security (health) that handles the professional and the client that uses it. The assets that promote the conformational change of the hairs are proteolytic enzymes and enzymatic activators agents that provide greater specificity in altering chemical bonds of the hair. The kit also has products to restore the properties of the capillary structure, evidenced by efficacy trials where attributes like hair shine, softness and strength were improved beyond protection against chemical and weathering. Thus, the kit is characterized by a complete treatment for the hair, since its preparation for further processing to recover conformational structures of fiber for maintaining the attributes mentioned above, besides providing protection against chemical and weathering by forming a protective film on the fibers.

The purpose of the Kit has been studied in each stage to be used as a complete treatment of conformational transformation of capillary fibers so as to act effectively and safely to the hair and scalp. The kit has been developed in conjunction with a method for the hairdresser professional apply for the well being of the client for the shortest time of application, and for greater security for the professional, being a product containing biotech assets, making it softer and more specific in their action.

### DETAILED DESCRIPTION OF THE INVENTION

The kit was specifically designed to be used in extremely curly and brittle hair as the african hair with efficacy and safety. The use of the complete treatment of the kit by acting specifically on the disulfide bonds of hair keratin and to permit the restoration of the capillary integrity, can be safely used in hair periodically to maintain the capillary conformation, with the advantage of the use of other hair products such as dyes.

According to the present invention, the kit has six products, namely: Cleansing Shampoo, Transformation Cream (Base cream), Enzymatic Gel, Neutralizer, Conditioner and Finishing Fluid.

The cleansing shampoo, transformation cream (base cream); enzymatic gel; conditioner, and Finisher fluid have been specially formulated to allow the conformation modifying actives operate efficiently and smoothly while allowing maintenance of the attributes of a healthy hair, such as brightness, softness and tensile mechanical resistance. In relation to cleansing shampoo, it has a variant pH between 7.5 to 8.5 to open the cuticles of the hair by using pH modifying agent aminomethylpropanol. The surfactants used are sodium lauryl ether sulphate with a concentration between 20 to 35% by weight, cocamidopropyl betaine with a concentration between 1 and 10% by weight, diethanolamide at a concentration of 2.5 to 5.0% by weight, in addition of emulsifiers polymers and viscosity agents as glycerides caprylic/capric and tristearate-polyglyceryl-2 in a concentration of from 1.0 to 3.0% by weight. A cleaning agent of natural origin was used to enhance the detergency effect on the hair called Pelvetia canaliculata extract in concentrations between 0.05 and 1.5% by weight. It was also used preservative diazolinil urea, propylene glycol and lodopropynyl butylcarbamate in a concentration between 0.2 to 0.4% by weight.

The Transformation Cream has enzymatic activators as calcium hydroxide and sodium sulfide in a concentration from 0.05 to 8% by weight, the reducing agent triethanolamine or ammonium thioglycolate at a concentration of 0.01 to 10% by weight. Also emulsifiers used are cetyl alcohol, glyceryl stearate, cetoestearate 20, sodium acrylates copolymer, hydrogenated polydecene, sorbitan trioleate, glyceryl dibehenate, and tribehenyl behenate glyceryl in concentrations ranging from 0.1 to 3.0% by weight. It was also used as preservative the diazolinil urea, propylene glycol and lodopropynyl butylcarbamate in a concentration between 0.2 to 0.4% by weight. The pH of the cream is kept within a threshold from 8.0 to 9.0.

The gel containing enzymes, has a pH around 7.5 to 8.5 with the aid of glycine-HCl buffer with a pH between 9.0 and 10.0 and concentration of between 0.2 to 0.4 mol/dm³. It is used as a viscosity agent an acrylate in a concentration between 0.7 and 1.5% by weight. The enzymatic activator cysteine hydrochloride is used at concentrations from 0.01 to 3.0% by weight, plus cysteine concentration of 0.01 to 2.0% by weight. The proteolytic enzymes used are: EC: 3.4.21.14—CAS# 9014-01-1 INS1101 i (Protemax® 580 L) in a concentration of from 0.01 to 3% by weight, EC: 3.4.21.62—CAS# 9014-01-1 and INS1101 I (Prolav® 750) in a concentration of 0.01 to 3% by weight and, EC: 3.4.24.28—CAS# 76774-43! NS1101 i (Protemax® NL600C) with a concentration between 0.01 to 3 wt %, presented in their trade names provided by the company Prozyn Indústria e Comércio Ltda. It was also used as a preservative the diazolynyl urea, propylene glycol and lodopropynyl butylcarbamate in concentration from 0.2 to 0.4% by weight. The product mixture is in the proportion 1:2 gel and cream activator, respectively. The neutralizer used in the kit is a commercial product found commonly at the market (H₂O₂ 20 volumes or 6% by weight) and has the function of finish the reduction reactions.

The repair conditioner consists of conditioning agents and emollients. Comprising the formulation soybean oil, corn and flowers oil, as well as jojoba esters, rice, candelilla, polyisobutene, stearates, cetostearates, sodium stearoyl lactylate, hydrolyzed keratin, aminodimethicone, dimethicone, polyquaternium 55, cyclopentasyloxane laroyl lysine, glycine, and wetting agents such as glycerin and PCA-Na.

The Finishing Fluid is compound of hydrophobic silicons that make a kind of deposition on the capillary surface. It has been used cyclomethicone, aminodimeticone aminopropyl phenyl trimethicone in varying concentrations from 2.0 to 10% by weight, with the function of thermal protection against the action of weather (humidity, wind, dust, and solar radiation) and chemical protection (chloro, pollutants and dyes).

The cosmetic kit for use in a conformational change of hair (straightening or curly has a pre-shampoo treatment with appropriate pH (basic) to facilitate the permeation of the actives that will modify the chemical bonds that will change the fibers. The kit also features two products that work combined and are responsible for the conformational change of the hair fibers. One of these products is in gel form and contains proteolytic enzymes and the other is in the form of a cream and has enzymatic activators agents. The resulting mixed product should be applied to relatively short hair size for a reaction time during 20 to 25 min for african hair and shorter times for other types of hair and reaction temperature around 40 to 45° C., held for thermal cap. After applying the hair are manipulated according to the necessity of maintaining conformational with the use of styling hair curling in the process of curling or the use of extenders to stretch the hair and in the case of smoothing. The enzyme activity is also maintained inert by the presentation of a cream containing the activating enzyme and the gel containing enzymes in separate packages to ensure the shelf life of the products. The design of the activating enzyme in the form of a cream facilitates the application of the product in professional hair due to product consistency and the ability to preview the application in each quadrant of the head. Subsequently the hair are washed and neutralized with oxidizing agent and treated with another product of Cosmetic Kit that has a slightly acid pH and agents to seal the cuticles (plant extracts, silicones, polymers) and so restore hair structure. Subsequently the hair are dried with dryer, and after this step is applied finisher agent with intention to form a film by the action of hydrophobic agents on the hair fibers.

### Method of Application for Kit:

A) Wash hair with the cleansing shampoo for the preparation of hair transformation. Wash with water for removal of the product before application of the transformation cream processing;

B) Remove the excess of humidity with towel;

C) Preparation of Transformation Activated Cream, which is the mixing of the contents of the Gel with Transformation Cream to activate the enzymes, homogenizing well. This mixture conforms to 2:1, respectively;

D) Apply lock to lock with a brush the Activated Cream Transformation, respecting the divisions in the four quadrants of the head. To have an action of straightening of the hair, stretch the hair by applying manual force and also the comb when possible, towards the root-tip of the lock;

E) Cover the head with a wet towel on the treated hair, and put a thermal cap. Let sit 20 to 25 minutes, for African hair. Caucasian hair or just slightly curly hair the time of contact with the product should be from 10- to 15 minutes;

F) After the period of action of the product, wash thoroughly with warm water to remove the total product. Remove the excess with a towel;

G) Apply the neutralizing and let act for 3 to 5 minutes;

H) Rinse hair again to remove the neutralizer and apply conditioner throughout the hair. Leave it for 2 to 4 minutes massaging the wires;

I) Rinse with water and dry your hair with towel, removing the excess well (Humidity);

J) Dry with a hair dryer on medium heat, 80 to 100° C.;

K) Apply the Finishing Fluid through the wires to the tip by hand, removing the excess of the hands on top of head and side;

L) Make the brush through hair and finish with the board thermal lock to lock, to transform smoothing;

M) It is necessary to inform the client to not wash their hair for a period of 24 hours after product application;

If instead straightening, it would prefer to curly the hair, performing the steps A to D and further tangling of hair curlers or through bobs. Just execute the steps from E to J to finish the process. Before the product application, the Activated Transformation Cream (the mixture of Transformation Cream and the Enzymatic Gel) should be used in a lock of hair to be evaluated, preferably without the neck hair and wait for 15 to 20 minutes to check the resistance of the hair product.

The product must not be applied in hair straightening that suffered less than eight months with products containing guanidine.

Still as regards the method, the hairs are manipulated according to the need of maintaining conformational using curl styling to the hair, permanently or in case of use of extenders to stretch the hair and in the case of smoothing. The gel containing enzymatic activators and enzymes are presented in separate packages to ensure enzyme activity to be maintained in inert formulation in order to guarantee the shelf life of the products. The design of enzymatic activators in cream form facilitates the application of the product on the hair by the professional because of its consistency and by viewing the application in each quadrant of the head. Subsequently the hair is washed and neutralized with oxidizing agent and treated with another product of Cosmetic Kit that has a slightly acid pH and agents to seal the cuticles (plant extracts, silicones, polymers) and so restore the hair structure. Subsequently the hair is dried with dryer, and after this step, the finisher agent is applied with the purpose of forming a film by the action of hydrophobic agents on the hair fibers.

1. COSMETIC KIT, COMPOSITION FOR CHANGING THE FORM OF HAIR FIBRES AND CORRESPONDING APPLICATION METHOD, characterized by being composed of:
Cleanser Shampoo with a pH variant between 7.5 to 8.5, using surfactants like sodium lauryl ether sulphate with a concentration between 20 to 35% by weight, and cocamidopropyl betaine with a concentration between 1 and 10 wt % acid diethanolamide coconut fatty acid concentration from 2.5 to 5.0% by weight addition polymer emulsifying agents and viscosity glycerides of caprylic/capric and tristearate-polyglyceryl-2 concentration between 1.0 and 3.0% by weight;
Transformer Cream (cream base) compound of enzyme activating agents, preferably cysteine hydrochloride in a concentration of from 0.01 to 3.0% by weight, plus cysteine concentration of 0.01 to 2.0% by weight;
Gel compound of proteolytic enzymes EC: 3.4.21.14—CAS# 9014-01-1 INS1101 I in concentrations from 0.01 to 3% by weight, EC: 3.4.21.62—CAS# 9014-01-1 in a concentration between 0.01 to 3% by weight, EC: 3.4.24.28 INS1101 i—CAS# 76774-43-1 at concentrations from 0.01 to 3% by weight;
Neutralizer (H₂O₂ 20 volumes or 6% by weight);
The repair conditioner consists of conditioning agents and emollients. Comprising the formulation soybean oil, corn and flowers oil, as well as jojoba esters, rice, candelilla, polyisobutene, stearates, cetostearates, sodium stearoyl lactylate, hydrolyzed keratin, aminodimethicone, dimethicone, polyquaternium 55, cyclopentasyloxane laroyl lysine, glycine, and wetting agents such as glycerin and PCA-Na.
The Finishing Fluid is compound of hydrophobic silicons that make a kind of deposition on the capillary surface. It has been used cyclomethicone, aminodimeticone aminopropyl phenyl trimethicone in varying concentrations from 2.0 to 10% by weight, which is used in extremely curly hair and brittle or fragile flat and acting specifically on the conformational change of keratin, especially in their disulfide bonds, and processing the cream (cream base) must be applied to the gel along with conformational change.

2. COSMETIC KIT AND THEIR COMPOSITION, according to claim 1, characterized in shampoo containing a cleaning agent of natural origin known Pelvetia canaliculata extract in a concentration of from 0.5 to 5% by weight.

3. COSMETIC KIT AND THEIR COMPOSITION, according to claim 1, characterized in shampoo using a preserving agent diazolinil urea, propylene glycol and lodopropynyl butylcarbamate in a concentration between 0.2 to 0.4% by weight.

4. COSMETIC KIT AND THEIR COMPOSITION, according to claim 1, characterized by modifying the conformation capiliary is composed of the mixture of cream containing activating enzyme and a gel containing three proteolytic enzymes.

5. COSMETIC KIT AND THEIR COMPOSITION, according to claim 1, characterized in cream activator present enzymatic activators calcium hydroxide and sodium sulphite in a concentration between 0.05 and 8% by weight, the reducing agent thioglycolate in ammonium or triethanolamine concentration from 0.01 to 10% by weight.

6. COSMETIC KIT AND THEIR COMPOSITION, according to claim 1, wherein the Activator cream uses emulsifying agents, such as cetyl alcohol, glyceryl stearate, ceteth-20, sodium acrylates copolymer, hydrogenated polideceneo, sorbitan trioleate, glyceryl dibehenate, tribehenyl behenate and glyceryl in concentrations thresholds from 0.1 to 3.0% by weight.

7. COSMETIC KIT AND THEIR COMPOSITION, according to claim 1, characterized by the use of a Activator Cream using as preservative the diazolinyl urea, propylene glycol and lodopropynyl butylcarbamate in a concentration between 0.2 and 0.4% by weight, and the pH of the cream is kept between 8.0 to 9.0.

8. COSMETIC KIT AND THEIR COMPOSITION, according to claim 1, characterized by the enzymes present in the gel with a pH around 7.5 to 8.5 with the aid of glycine-HCl buffer with a pH threshold between 9.0 and 10 and 0.2 to 0.4 mol/dm³.

9. COSMETIC KIT AND THEIR COMPOSITION according to claim 1, characterized by the use of a gel that have in it composition a viscosity agent based on acrylate in a concentration between 0.7 and 1.5% by weight.

10. COSMETIC KIT AND THEIR COMPOSITION, according to claim 1, characterized by using a gel preservative diazolinil urea, propylene glycol and lodopropynyl butylcarbamate in a concentration between 0.2 to 0.4% by weight.

11. COSMETIC KIT AND THEIR COMPOSITION, according to claim 1, wherein the product mixture must be taken in the ratio 1:2 gel and cream activator, respectively.

12. COSMETIC KIT AND THEIR COMPOSITION, according to claim 1, wherein the agent conformational require 15 to 25 minutes to remain on the hair at temperatures around 40 to 45° C. with the aid of thermal cap and the hair must be extended and stretched in the case of smoothing and used “bobs” or other tools to curl the hair.

13. APPLICATION METHOD OF COSMETIC KIT AND IT COMPOSITION FOR CONFORMATIONAL CHANGE OF THE HAIR FIBERS, comprising the following steps:
A) Wash hair with the cleansing shampoo for the preparation of hair transformation. Wash with water for removal of the product before application of the transformation cream processing;
B) Remove the excess of humidity with towel;
C) Preparation of Transformation Activated Cream, which is the mixing of the contents of the Gel with Transformation Cream to activate the enzymes, homogenizing well. This mixture conforms to 2:1, respectively;
D) Apply lock to lock with a brush the Activated Cream Transformation, respecting the divisions in the four quadrants of the head. To have an action of straightening of the hair, stretch the hair by applying manual force and also the comb when possible, towards the root-tip of the lock;
E) Cover the head with a wet towel on the treated hair, and put a thermal cap. Let sit 20 to 25 minutes, for African hair. Caucasian hair or just slightly curly hair the time of contact with the product should be from 10- to 15 minutes;
F) After the period of action of the product, wash thoroughly with warm water to remove the total product and remove the excess with a towel;
G) Apply the neutralizing and let act for 3 to 5 minutes;
H) Rinse hair again to remove the neutralizer and apply conditioner throughout the hair, and leave it for 2 to 4 minutes massaging the wires;
I) Rinse with water and dry your hair with towel, removing the excess well (Humidity);
J) Dry with a hair dryer on medium heat, 80 to 100° C.;
K) Apply the Finishing Fluid through the wires to the tip by hand, removing the excess of the hands on top of head and side;
L) Make the brush through hair and finish with the board thermal lock to lock, to transform smoothing;
M) It is necessary to inform the client to not wash their hair for a period of 24 hours after product application.

14. APPLICATION METHOD OF COSMETIC KIT AND IT COMPOSITION FOR CONFORMATIONAL CHANGE OF THE HAIR FIBERS according to claim 13, characterized by the method of curly using steps A through D and promote tangling hair through “bobs” or “bigudins”, running up the steps from A to D and from E to J to finish the process.

15. APPLICATION METHOD OF COSMETIC KIT AND IT COMPOSITION FOR CONFORMATIONAL CHANGE OF THE HAIR FIBERS according to claim 13, characterized in that, before the product application, the Activated Transformation Cream (the mixture of Transformation Cream and the Enzymatic Gel) should be used in a lock of hair to be evaluated, preferably without the neck hair and wait for 15 to 20 minutes to check the resistance of the hair product.
